(19) United States
(12) Patent Application Publication
(10) Pub. No.: US 2005/0239051 A1
(43) Pub. Date: Oct. 27, 2005

(54) Use of isogenic human cancer cells for high-throughput screening and drug discovery

(75) Inventors: Christopher J. Torrance, Baltimore, MD (US); Bert Vogelstein, Baltimore, MD (US); Kenneth W. Kinzler, Bel Air, MD (US)

Correspondence Address: BANNER & WITCOFF, 1001 G STREET N W, SUITE 1100, WASHINGTON, DC, 20001, US

(73) Assignee: The Johns Hopkins University, Baltimore, MD (US)

(21) Appl. No.: 11/159,229

(22) Filed: Jun. 23, 2005

Related U.S. Application Data
(63) Continuation of application No. 09/961,407, filed on Sep. 25, 2001.

(51) Int. Cl. C12Q 1/00; C12Q 1/68; C12N 5/08

(52) U.S. Cl. 435/4; 435/366

(57) ABSTRACT

A strategy for drug-screening is based on cells that are isogenic except for a gene of interest. Each cell can be tranfected with a vector that encodes a different fluorescent protein that can be differentially detected to monitor cell growth. Co-culture of both cells allows facile screening for compounds with selective toxicity towards a gene of interest. The drug screening is broadly applicable for mining therapeutic agents targeted to specific genetic alterations responsible for cancer development.

This invention was made using funds from the U.S. National Institutes of Health (CA43460, CA09243, and CA62924). Therefore, the government retains some rights in the present invention.

### FIELD OF THE INVENTION

The invention relates to the area of high-throughput screening assays for therapeutic agents. More particularly, the invention relates to the area of screening assays for cancer drug discovery. Furthermore, the invention relates to compounds and methods of treating cancer.

### BACKGROUND OF THE INVENTION

The past fifteen years have witnessed an explosion of knowledge concerning the genetic basis of human cancer. This has raised enormous potential for developing novel therapeutics aimed at targeting the genetic differences that exist between tumor cells and all normal cells in the body. As yet, however, this potential has only been realized in a few tumor types with overexpressed or deregulated cellular oncogenes e.g., ST1571, an inhibitor of the c-ab1/c-kit tyrosine kinases (Druker et al., N. Engl. J. Med., 344:1038-42, 2001) and trastuzumab (Herceptin), a humanised monoclonal antibody which inhibits the human epidermal growth factor receptor-2 (HER-2/neu) (Pegram et al., Cancer Treat. Res., 103:57-75, 2000). Novel strategies that target genetic alterations such as these are desperately needed if the dream of improved therapeutics is to be realized.

Tissue culture cell-based screening for anti-cancer drugs has been used for decades. However, a persistent problem with such cell-based screening lies in the nature of the control cells. Many compounds are toxic to cancer cells, but most are also toxic to all growing cells. Normal cells corresponding to the cell types represented by common tumors are generally not available or do not exhibit growth properties comparable to those of the tumors. For these and other reasons, it is difficult to determine whether the drugs are targeting tumor-specific molecules. Many strategies have been employed to overcome this problem. For example, the National Cancer Institute (NCI) created the ‘In Vitro Cell Line Screening Project’ (IVCLSP) using a panel of 60 cancer cell lines in an attempt to correlate unique drug sensitivities with specific genetic alterations. The interpretation of the results achieved with this ambitious and useful approach, however, is limited by the numerous and largely uncharacterized somatic and inherited genetic differences between cancers from different individuals. In other cases, drug screens using transformed cells over-expressing high levels of various oncogenes have been used (Jenkins et al., Br. J. Cancer, 68:856-61, 1993; Corbley et al., Int. J. Cancer, 66:753-59, 1996). Such over-expression, however, can be associated with non-physiological properties of the transformed cells and does not necessarily reflect the events occurring in human tumors with endogenous mutations of the same genes. The latter criticism also holds for screens using revertant subclones of transformed cell lines (Stratowa et al., Anticancer Drug. Des., 14:393-402, 1999).

There is a need in the art for a drug screening strategy that exploits cells with defined, endogenous alterations of specific genes and provides for high-throughput, cost-effective screening of test compounds with selectivity towards any genetic alteration.

### BRIEF SUMMARY OF THE INVENTION

One embodiment of the invention is a pair of cells that are isogenic except for a gene of interest and a gene encoding a fluorescent protein. The first cell comprises a gene that encodes a first fluorescent protein having a first absorption spectrum and a first emission spectrum. The second cell comprises a gene that encodes a second fluorescent protein having a second absorption spectrum and a second emission spectrum. Either the absorption spectra of the first and second fluorescent proteins are not identical and/or the emission spectra of the first and second fluorescent proteins are not identical.

A second embodiment of the invention is a pair of cells that are isogenic except for the Ras genotype of the cells and a gene encoding a fluorescent protein. The Ras genotype of the first cell is c-Ki-Ras^{WT/mutant} and comprises a gene that encodes a first fluorescent protein having a first absorption and a first emisson spectrum. The Ras genotype of the second cell is c-Ki-Ras^{WT/null} and comprises a gene that encodes a second fluorescent protein having a second absorption and a second emission spectrum. The first and second absorption and emission spectra are not identical.

Another embodiment of the invention is a method of making a pair of cells by genetically modifying a first cell to yield a second cell that is isogenic except for a single gene of interest. The first cell is then transfected with a first gene that encodes a first fluorescent protein having a first absorption spectrum and a first emission spectrum. The second cell is transfected with a gene that encodes a second fluorescent protein having a second absorption spectrum and a second emission spectrum. Either the absorption spectra of the first and second fluorescent proteins are not identical and/or the emission spectra of the first and second fluorescent proteins are not identical.

Still another embodiment of the invention is a method of identifying a test compound as selectively affecting a gene of interest, its expression products, or downstream genes or proteins in its pathway. The method involves culturing a first cell and a second cell that are isogenic except for a gene of interest and a gene encoding a fluorescent protein. The first cell comprises a gene that encodes a first fluorescent protein having a first absorption spectrum and a first emission spectrum. The second cell comprises a gene that encodes a second fluorescent protein having a second absorption spectrum and a second emission spectrum. Either the absorption spectra of the first and second fluorescent proteins are not identical and/or the emission spectra of the first and second fluorescent proteins are not identical. The first and second cells are contacted with a test compound and the test compound is identified as selectively affecting the gene of interest, its expression products, or downstream genes or proteins in its pathway if the growth rate of the first cell is altered with respect to the second cell.

A further embodiment of the invention is a method of identifying a test compound as selectively affecting a Ras gene, Ras protein, or downstream gene or protein in its pathway. First and second cells that are isogenic except for their Ras gene and a gene encoding a fluorescent protein are contacted with a test compound. The Ras genotype of the first cell is c-Ki-Ras^{WT/mutant} and the first cell further comprises a first gene encoding a first fluorescent protein having a first absorption spectrum and a first emission spectrum. The Ras genotype of the second cell is c-Ki-Ras^{WT/null} and the second cell further comprises a second gene that encodes a second fluorescent protein having a second absorption spectrum and second emission spectrum. A test compound is identified as selectively affecting the Ras gene, Ras protein, or downstream gene or protein in the pathway if the growth rate of the first cell is altered with respect to the growth rate of the second cell.

Another embodiment of the invention is a composition comprising at least 90% of a cytidine analog having the formula: 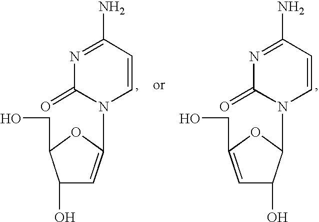
or a mixture thereof.

Even another embodiment of the invention is a pharmaceutical composition comprising a compound having the formula: 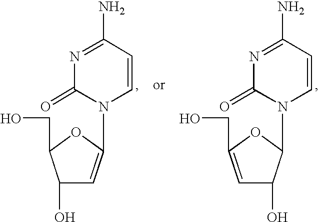
or a mixture thereof and a pharmaceutically appropriate carrier.

Still another embodiment of the invention is a cytotoxic composition comprising triphenyltetrazolium (TPT) and a pharmaceutically appropriate carrier.

Yet another embodiment of the invention is a method of treating cancer comprising administering to a patient in need thereof a therapeutically effective amount of a compound having the formula 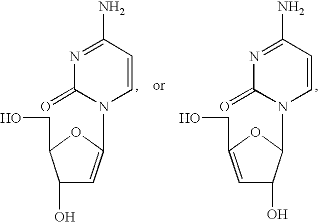
or a mixture thereof and a pharmaceutically appropriate carrier.

These and other embodiments of the invention provide the art with tools and methods for high throughput screening of therapeutic compounds for drug discovery. Additionally, embodiments of the invention provide the art with compounds for the treatment of cancer.

### BRIEF DESCRIPTION OF THE DRAWINGS

FIG. 1. Schematic of drug screening format using co-cultured isogenic cells modified with spectrally distinct green fluorescent protein (GFP) variants. DLD-1 cells, harbouring a mutant c-Ki-RAS allele plus a wild-type (wt) c-Ki-RAS allele, were modified to stably express a GFP variant that is excited at 530 nm (YFP). An isogenic clone (KO) in which only the mutant c-Ki-RAS allele has been deleted (Adegboyega et al., Arch. Pathol. Lab. Med., 121:1063-68, 1997) was modified to express a GFP variant that is excited at 390 nm (BFP). These cells were mixed and plated at low density in 96-well plates. After an overnight incubation to allow cell attachment, test compounds are added in 50 μl of culture media to a final concentration of 2 μg/ml. Plates were assayed every day for six days using a fluorescence plate reader to obtain growth curves for each cell line in every well of the plate.

FIG. 2. Structures of SC-D (a) and TPT (b).

FIG. 3. Fluorescent micrographs showing selective growth inhibition of DLD-1 cells co-cultured with KO cells by two test compounds. Left panels=DLD-1 cells excited using blue light (Nikon filter UV-2A); right panels=KO cells excited using yellow light (Omega Optical filter XF104). Top panels depict untreated controls, 6 days after plating cells. Lower panels depict cells six days after plating in the presence of drug.

FIG. 4a. A representative 96-well plate showing primary data from the DLD-1/KO drug screen. Column 1=no cell controls for subtracting the background fluorescence of the culture media; Column 12=no-drug control (blue line=DLD-1; red line=KO). A total of 80 compounds/plate were tested in Columns 2-11. The average of the no-drug controls (Column 12) were plotted in every window of the 96 well plate for facile visualization of compounds that caused growth inhibition. The x-axis of each graph denotes hours after adding drugs (0-150 hrs). Δ=KO cells, no drug; ▴=KO cells+drug; ⋄=DLD-1 cells, no drug; ♦=DLD-1 cells+drug.

FIG. 4b. Well #R6C4 (Row 6, Column 4 in FIG. 4a) demonstrates a compound with selective toxicity for DLD-1. Well #R7C4 demonstrates a compound with toxic effects on both DLD-1 and KO cells. The majority of wells contain compounds with no discemable effect on cell growth compared to controls e.g., R6C5 and R7C5. The x-axis of each graph denotes hours after adding drugs (0-150 hrs). Δ=KO cells, no drug; ▴=KO cells+drug; ⋄=DLD-1 cells, no drug; ♦=DLD-1 cells+drug.

FIG. 5. Growth curves of co-cultured DLD-1 and KO cells exposed to increasing concentrations of the test compounds SC-D and TPT. Growth curves were obtained from fluorescence at 390ex/508em (DLD-1) and 530ex/590em (KO). The x-axis in each graph denotes days after drug addition.

FIG. 6. Structure/activity ratios (SAR's) of compounds with structural homology to TPT. Growth inhibition assays were performed using parental (non-GFP modified) cell lines. Cells were plated separately at low density in 96 well plates and exposed to a range of test drug concentrations for five days. Cells were then lysed in H₂O, and lysates assayed for relative cell number using the dsDNA-binding dye (PicoGreen).

FIGS. 7A and 7B. Effect of SC-D on tumor xenografts. FIG. 7A shows colon cancer HCT116, treated with SC-D (300 mg/kg). FIG. 7B shows colon cancer DLD-1 treated with SC-D (150 mg/kg). Relative tumor volume=average tumor volume (T)/average starting tumor volume (S).

### DETAILED DESCRIPTION OF THE INVENTION

It is a discovery of the present invention that cells that are isogenic except for a gene of interest are particularly useful for identifying compounds that specifically affect that gene, its expression products, or downstream genes or proteins in its pathway. The development of agents that specifically target a specific genotype can lead to more efficacious and less toxic therapeutic agents.

The present invention provides a drug-screen that exploits human cancer cells with defined, endogenous alterations of specific genes. By altering one gene in such cells an isogenic clone can be created which differs from its parent only in this single gene. In this way, one can directly screen for compounds with selective toxicity towards any genetic alteration. The invention also provides a screening strategy involving co-culture of parental and targeted cells, thus allowing precise internal calibration of each drug assay and rapid throughput (˜12,000 compounds/week).

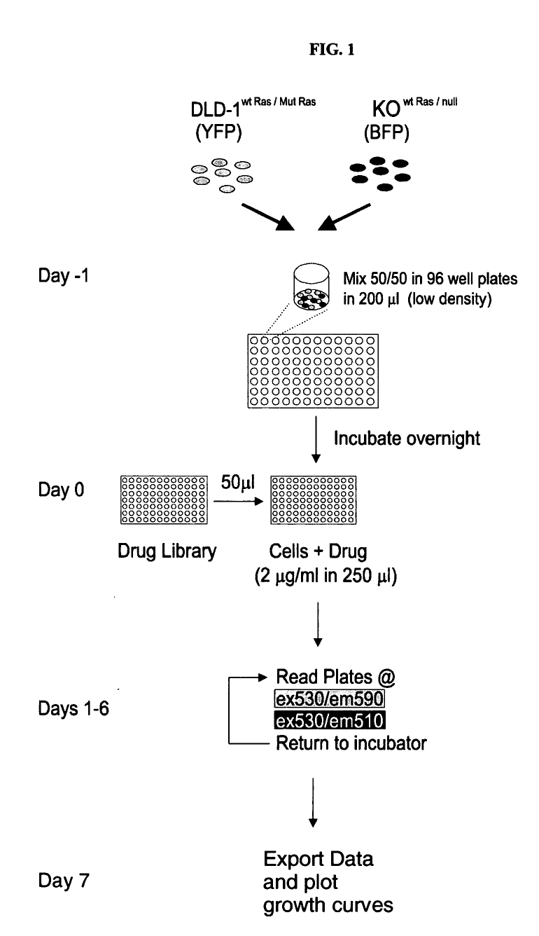

The strategy of the present invention involves two components (see FIG. 1). The first component is the use of paired cells that differ in a single gene that is altered in one of the two cells. The second component involves genes that encode different fluorescent proteins in each of these cells, thus individually marking the cells to allow for differential detection. The cells are then cultured, as a co-culture or individually, and their growth rates followed using a fluorescence spectroscopic technique.

The present invention provides several major advantages over drug screening techniques in common use. Because the two cells to be compared can be co-cultured and assayed simultaneously a variety of errors encountered when screening cell pairs that are maintained in separate compartments are eliminated. Second, cell growth patterns can be followed over time and the early time points can serve as internal controls for each well, normalizing for variability of cell numbers across samples as well as for the inherent fluorescence of certain drugs. Finally, assays involving engineered fluorescence proteins are highly cost-effective, because no additional reagents such as luciferase or pipetting steps are required for analysis of growth (Kain, Drug Discov. Today, 4:304-12, 1999).

A strategy for drug-screening that exploits human cancer cells with defined, endogenous alterations of specific genes has numerous advantages. Using an isogenic clone that differs from its parent in a single mutant gene, one can directly screen for compounds with selective toxicity towards any genetic alteration. The screening procedure described here will extend the utility of somatic knock-outs beyond basic mechanistic studies and provide a means to rationally discover lead compounds targeted to specific genes and the pathways they control.

It must be noted that as used herein, the singular forms “a,” “an,” and “the” include plural reference unless the context clearly dictates otherwise. Thus, for example, a reference to “a cell” includes a plurality of such cells.

Isogenic Cells Differing in a Single Gene of Interest

The term “isogenic,” as it is used here, refers to the cell's own endogenous genome. Any type of mammalian cell that can be maintained in a culture can be transfected and used to generate a cell with specific genetic alterations. These cells include, but are not limited to, primary cells, such as fibroblasts, myoblasts, leukocytes, hepatocytes, endothelial cells, and dendritic cells, as well as cell lines (e.g., NCI-BL2126, Hs 578Bst, HCC1954 BL, Hs 574.Sk, Hs888Lu, which are available from the American Type Culture Collection, 10801 University Boulevard, Manassas, Va. 20110-2209). Established tumor cell lines, such as HT29, SW480, HCT116, DLD1, MCF-7, HL-60, HeLa cell S3, K562, MOLT-4, Burkitt's lymphoma Raji, A549, G361, M12, M24, M101, SK-MEL, U-87 MG, U-118 MG, CCF-STTG1, or SW1088 can be used. Preferred cells include human cells, preferably human tumor cells, more preferably human colon tumor cells and human breast tumor cells.

For purposes of the present invention, a cell with its normal complement of genes is “wild-type.” Any alteration in at least one endogenous gene is considered a mutant. Any defined, endogenous alteration of specific genes may be employed. While not an exhaustive list, the gene of interest may vary between two cells as a substitution in a single amino acid in the gene product, a deletion of at least one amino acid in the gene product, the level of gene expression may be reduced or increased, or the gene may not be expressed at all. Preferably, the genetic alteration renders the cell tumorigenic in a mouse model system. Preferably, the pair of wild-type and mutant cells used in methods of the invention are of the same type of cell (i.e., originates from the same type of tissue and organ). More preferably, the two cells are isogenic except for a single gene of interest.

Any means known in the art to create cells with defined alterations in specific genes may be used. For example, homologous recombination can be used to generate an isogenic clone that differs from its parent cell only in a single mutant gene (Capecchi, Science, 244:1288-92, 1989).

A pair of cells can be in a single vessel, or a divided vessel, including, without limitation, a cell culture dish or flask, a multi-well cell culture plate, a liquid nitrogen container, a freezer box, a freezer, a refrigerator, a tissue culture hood, or an analytical device.

Cells Comprising Fluorescent Proteins

The isogenic cells described above can contain a gene that encodes a fluorescent protein. All proteins capable of detection by fluorescence techniques are contemplated, including variants of known fluorescent proteins such as mutants of green fluorescent protein that have been modified to alter the absorbance and emission spectra of the protein (see, e.g. Prasher et al., Gene, 111:229-33, 1992; Chalfie, Photochem. Photobiol., 62:651-6, 1995; Miteli & Spector, Nature Biotechnol., 15:961, 1997; Heim, et al., Nature 373:663-4, 1995; Cubitt, et al., Trends Biochem., 20:448-55, 1995. Examples of suitable fluorescent proteins include green fluorescent protein (395 nm absorption/509 nm emission), yellow fluorescent protein (530 nm absorption/590 nm emission), red fluorescent protein, cyan fluorescent protein (433 nm absorption/475 nm emission) and blue fluorescent protein (390 nm absorption/510 nm emission) (Clonetech Living Colors® User Manual, August 2000).

The gene may be integrated in the genome, or more preferably, on an expression vector. Any expression vector encoding a protein capable of detection by fluorescence spectroscopy is contemplated. Useful fluorescent protein vectors include, but are not limited to pGFPtpz-cmv and pGFPsph-cmv (Packard Instrument Co.) as well as pGFP, pEGFP, pEBFP, pEYFP, pECFP, and pd2EGFP (Clonetech Laboratories, Inc.). Expression vectors may be further modified to include selectable markers, such as antibiotic resistance genes. Preferably progeny of a single transfection will exhibit uniform fluorescent protein expression.

An appropriate expression vector contains the necessary elements for the transcription and translation of the inserted coding sequence in a given cell type. Methods which are well known to those skilled in the art can be used to construct expression vectors containing sequences encoding fluorescent proteins and appropriate transcriptional and translational control elements. Such techniques are described, for example, in Sambrook et al., Molecular Cloning: A Laboratory Manual, 2d. ed., Cold Spring Harbor Laboratory Press, Cold Spring Harbor, N.Y., 1989, and in Ausubel et al., Current Protocols in Molecular Biology, John Wiley & Sons, New York, N.Y., 1989.

Depending upon the vector system and host utilized, any number of suitable transcription and translation elements, including constitutive and inducible promoters, can be used. In mammalian cell systems, promoters from mammalian genes or from mammalian viruses are preferable. A number of viral-based expression systems can be used to express fluorescent proteins in mammalian host cells. For example, if an adenovirus is used as an expression vector, sequences encoding fluorescent proteins can be ligated into an adenovirus transcription/translation complex comprising the late promoter and tripartite leader sequence. Insertion in a non-essential E1 or E3 region of the viral genome can be used to obtain a viable virus which is capable of expressing fluorescent proteins in infected cells (Logan & Shenk, Proc. Natl. Acad. Sci., 81:3655-3659, 1984. If desired, transcription enhancers, such as the Rous sarcoma virus (RSV) enhancer, can be used to increase expression in mammalian cells.

Any means known in the art to transfect cells with expression vectors may be used. These methods include, but are not limited to, transferrin-polycation-mediated DNA transfer, transfer with naked or encapsulated nucleic acids, liposome-mediated cell fusion, intracellular uptake of DNA-coated latex beads, protoplast fusion, viral infection, electroporation, and calcium phosphate-mediated transfection. Integration of the DNA sequences encoding the a fluorescent protein into the host cell's DNA can be facilitated by providing nucleotides at the 3′ or 5′ ends of these DNA sequences which are homologous to and therefore recombine with the host cell DNA. One or more copies of each DNA sequence can be integrated into the genome of the cell, as desired.

Any method known in the art for detecting a fluorescent protein is contemplated. The detection method may be, though need not be, quantitative. Fluorescent proteins can be detected by flow cytometry and fluorimetic assays. Preferably, fluorescent proteins are detected using fluorescence microscopy or high throughput fluorescence spectroscopy. More preferably, relative growth of the cells is followed in real time using fluorescence spectroscopy.

Culturing Cells

Cells can be co-cultured or cultured separately. When cells are co-cultured, each cell preferably contains a different fluorescent protein. To facilitate detection, the fluorescent proteins should have absorption and/or emission spectra that allow the proteins to be distinguishable in the detection method selected. It should be possible to either selectively excite the fluorescent protein or to selectively detect the emission of the fluorescent protein. The fluorescent proteins should either have absorption spectra that are not identical or emission spectra that are not identical or both. Preferably, the fluorescent proteins will have either absorption or emission spectra that are non-overlapping. More preferably, the fluorescent proteins will have non-overlapping absorption and emission spectra.

Methods of Screening Test Compounds

Cells that are isogenic except for a gene of interest and a gene that encodes a fluorescent protein can be used to screen test compounds for the ability to specifically affect the gene of interest. The cells are contacted with a test compound and the growth rates of the cells are monitored both before contact with the test compound and after contact with the test compound. A test compound that changes the growth rate of the first cell relative to the second cell is identified as selectively affecting the gene of interest.

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is typically used for polypeptide libraries, while the other four approaches are applicable to polypeptide, non-peptide oligomer, or small molecule libraries of compounds. See Lam, Anticancer Drug Des., 12:145, 1997.

Test compounds may be screened for the ability to slow the growth of cells possessing defined, known genetic alterations, including oncogenic mutations. More particularly, cells that are isogenic except for a gene of interest may be contacted with a test compound and the relative growth rates of the cells determined. To identify a test compound as selectively affecting expression of a gene of interest a pair of cells may be individually cultured or co-cultured in a single vessel using any appropriate cell growth medium and any appropriate growth vessel. Preferably, cells are co-cultured to allow precise internal calibration of each drug assay, eliminating a variety of errors encountered when screening cell pairs that are maintained in separate plates. An “essentially equal number” refers to literally equal (i.e. 10 of each of a first and second cell) and approximately equal (i.e. 1 of a first cell and 10 of a second, 1 of a first cell and 5 of a second, 1 of a first cell and 2 of a second, or 1 of a first cell and 1.5 of a second) and is defined as constrained to a less than or equal to 10-fold difference in the number of first and second cells present in a sample. It is necessary to know the relative amounts of each type of cell so that relative growth rates of the cells can be determined.

The isogenic cells can be grown and monitored for a period of time to obtain baseline growth rates prior to contacting the cells with a test compound. Growth rates of the cells can be determined qualitatively or quantitatively. Cell growth may be monitored by any means known in the art. Preferably, cells are monitored using a fluorescence spectroscopic technique. More preferably, cells are co-cultured and fluorescent proteins capable of being differentially detected are monitored simultaneously to obtain cell growth data. Any means to construct cell growth curves is contemplated.

Growth can be monitored using any fluorescence technique known in the art, as discussed above. A test compound may be supplied in any appropriate carrier or solvent and may be added to the cell culture by any means known in the art. Suitable solvents include, but are not limited to, aqueous solvents and DMSO. The growth rate of each cell in the pair of cells is then monitored in the presence of the test compound. The concentration of test compound used will vary widely depending on the compound and the conditions of the assay.

A test compound can be identified as selectively affecting a cell having a genotype of interest if the growth rate of a first cell possessing the genotype of interest is decreased relative to a second cell that does not possess the genotype of interest. Preferably, the test compound results in a differential change in the growth rate between the first and second cells of at least 25, 50, 75, 85, 90, 95, or 100 percent. More preferably, a test compound is capable of inhibiting cell growth in a cell having an oncogenic mutation such that the response exceeds the standard criteria for promising lead compounds established by the NCI (Geran et al., Cancer Chemother. Rep., 3:1-103, 1972).

Using high throughput screening, many discrete test compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format. Other formats can be used as are convenient.

Pharmaceutical Compositions

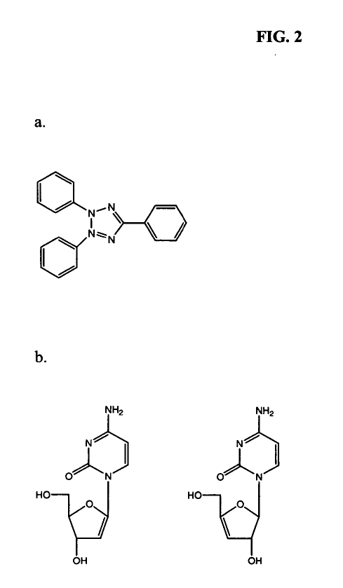

The invention also provides pharmaceutical compositions. One is Triphenyltetrazolium (TPT) (FIG. 2a) which can be purchased from Aldrich Chemical Company (Milwaukee, Wis.). Another is cytidine derivatives having an unsaturated sugar moiety (or sulfinyl cytidine derivative, “SC-D”) (FIG. 2b). SC-D can be synthesized by any method contemplated by the art and as described in Example 3. Included in the TPT and SC-D compounds shown in FIG. 2 are the stereoisomers thereof, the pharmaceutically-acceptable salts thereof, the tautomers thereof, and the prodrugs thereof.

The stereoisomers of the compounds may include, but are not limited to, enantiomers, diastereomers, racemic mixtures and combinations thereof. Such stereoisomers can be prepared and separated using conventional techniques, either by reacting enantiomeric starting materials, or by separating isomers of compounds of the present invention. Isomers may include geometric isomers. Examples of geometric isomers includes, but are not limited to, cis isomers or trans isomers across a double bond. Other isomers are contemplated among the compounds of the present invention. The isomers may be used either in pure form or in admixture with other isomers of the compounds described above.

Pharmaceutically acceptable salts of the compounds of the present invention include salts commonly used to form alkali metal salts or form addition salts of free acids or free bases. The nature of the salt is not critical, provided that it is pharmaceutically-acceptable.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyhnethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents that increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms.

Further details on techniques for formulation and administration are discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999; U.S. Pharamacopeia (Twenty-First Revision—USP XXI) National Formulary (Sixteenth Edition—XVI), United States Pharmacopeial Convention, Inc., Rockville, Md., 1985, and its later editions; and Remington's Pharmaceutical Sciences, 16^{th} Edition, Arthur Osol, Editor and Chairman of the Editorial Board, Mack Publishing Co., Easton, Pa., 1980, and its later editions.

The SC-D compounds of the present invention can be used to treat cancer and may be administered by any suitable route and in a therapeutically effective dose for the treatment intended. The active compounds and compositions, for example, may be administered orally, sublingually, nasally, pulmonarily, mucosally, parenterally, intravascularly, intraperitoneally, subcutaneously, intramuscularly or topically. The determination of a therapeutically effective dose is well within the capability of those skilled in the art. A therapeutically effective dose is that amount sufficient to reduce symptoms, reduce tumor size, reduce the rate of cell proliferation, or prevent metastasis.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Guidance as to particular dosages and methods of delivery is provided in the literature and generally available to practitioners in the art.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles.

Any of the therapeutic methods described above can be applied to any subject in need of such therapy, including, for example, mammals such as dogs, cats, cows, horses, rabbits, monkeys, and most preferably, humans.

TPT can be used to treat cancer and may be administered by any suitable route and in a therapeutically effective dose for the treatment intended. The active compounds and compositions may be administered by any means as discussed above with respect to SC-D. Determination of a therapeutically effective dose can also be accomplished as discussed for SC-D. A therapeutically effective dose is that amount sufficient to reduce symptoms, reduce tumor size, reduce the rate of cancer cell growth, or prevent metastasis.

While the invention has been described with respect to specific examples including presently preferred modes of carrying out the invention, those skilled in the art will appreciate that there are numerous variations and permutations of the above described systems and techniques that fall within the spirit and scope of the invention as set forth in the claims.

### EXAMPLE 1

### Drug Screening in DLD-1 and KO Colon Cancer Cell Lines

The drug-screening strategy of the present invention was implemented using DLD-1 cells in which the mutant c-Ki-Ras allele was deleted by targeted homologous integration (Shirasawa et al., Science, 260:85-8, 1993). Green fluorescent protein plasmid vectors, pGFPtpz-cmv (yellow fluorescent protein, YFP) and pGFPsph-cmv (blue fluorescent protein, BFP), were purchased from Packard Instrument Company (Meridian, Conn.). These vectors were further modified for drug selection in mammalian cells by insertion of the zeocin gene obtained from the plasmid pCMVzeo (Invitrogen, Carlsbad, Calif.). Isogenic cell lines DLD- 1 (Ki-Ras^{WT}/K-Ras^{G12D Mut}) and DKS-8 (Ki-Ras^{WT/Null}) were generously provided by S. Shirasawa, T. Sasauki and colleagues (Shirasawa et al., Science, 260:85-8, 1993). Clones with stable expression of the BFP vector in DLD-1 and the YFP vector in DKS-8 (herein designated as KO) were isolated by limiting dilution under zeocin selection (0.5 mg/ml). A single clone of each cell line was chosen for drug screening based on bright and uniform fluorescent protein expression.

A collection of 29,440 diverse small molecules were obtained from either Chembridge Corporation (DiverSet library) (San Diego, Calif.) or the National Cancer Institutes Developmental Therapeutic Program (Bethesda, Md.). All libraries were provided as DMSO solutions formatted in 96 well plates for easy transfer to cells. The p38 stress kinase inhibitor SB203580 was obtained from Promega (Madison, Wis.). Triphenyltetrazolium (TPT) was purchased from Aldrich Chemical Company (Milwaukee, Wis.). SC-D was synthesized as described in Example 3.

Fluorescent cell lines were harvested by trypsinization and resuspended together as a mixture containing 22,000 cells/ml (DLD-1) and 18,000 cells/ml (KO) in DMEM media supplemented with 10% FCS (HyClone, Logan, Utah). Approximately 8000 cells in a total of 200 μl were aliquoted into each well of 96 well plates for drug screening. Each well of column 1 contained 200 ml of the growth medium without cells as a control for computing background fluorescence. Plates were incubated overnight at 37° C. at 5% CO₂ in a humidified incubator. The following day, 50 μl of drug-containing medium was added to each well of columns 2 to 11. Each well of column 12 was designated a no-drug control and along with column 1 received 50 μl media without drug+DMSO. A total of 80 drugs were therefore tested in each plate. Final drug concentrations were 2 μg/ml for the Chembridge library and 2 μM for compounds obtained from the NCI. After drug addition, and each subsequent day for 6 days, plates were subjected to fluorometry to determine the growth curves for each cell line. DLD-1 and KO cells could be distinguished independently by the non-overlapping absorption and emission characteristics of their respective fluorescent proteins (BFP: 390 nm absorption/510 nm emission; YFP: 530 nm absorption/590 nm emission). Fluorometry data from each day's read were exported to a custom program (Drugmobile) for plotting the growth curves of each cell line in each well of the 96-well plate. Wells containing DLD-1 growth curves <2 standard deviations compared to the no-drug control average were scored as ‘hits’. Scoring hits in this fashion included compounds with selective toxicity towards DLD-1 cells, plus those which inhibited both DLD-1 and KO cells.

Secondary screens on primary screen hits were performed as above except that a drug concentration range was used, typically extending from 30 ng/ml to 4 μg/ml. Compounds found to be at least two-fold selective for DLD-1 versus KO cells in the secondary screen were then retested against other clones of the two cell types, as well as against clones that did not express fluorescent proteins. Assays using non-GFP modified cell lines were performed using cells plated separately rather than co-cultured. Plates were harvested each day for assessment of cell number using either an MTT assay or using the DNA-binding dye PicoGreen (Molecular Probes, Eugene, Oreg.). MTT assays were performed according to the manufacturers instructions (Trevigen, Gaithersburg, Md.). PicoGreen assays were performed by lysing cells in 96-well plates in 200 μl H₂O, triturating and then transferring a 10 μl aliquot from each well to 190 ml of PigoGreen solution (# P-7581) diluted to 0.5% vol/vol in Tris-EDTA (TE) buffer.

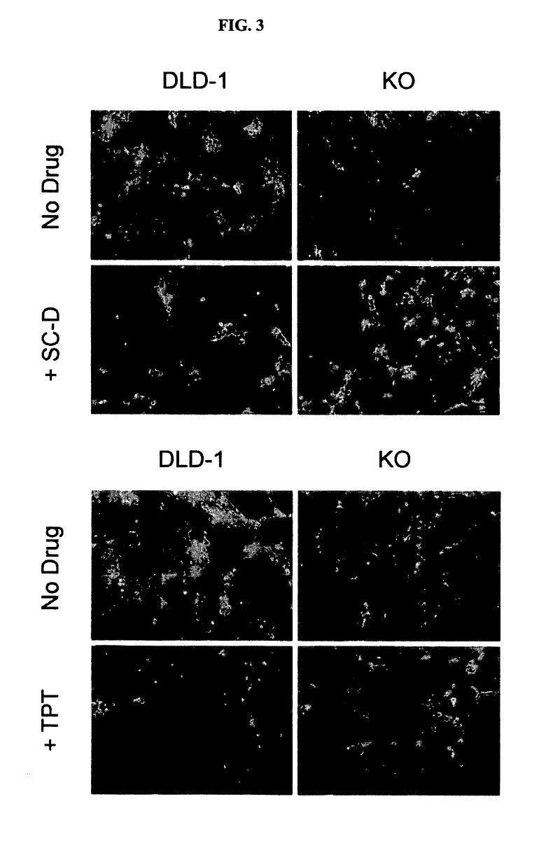

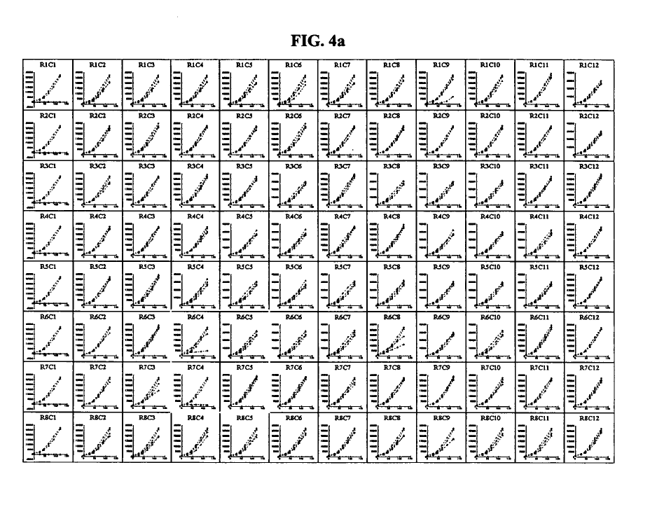

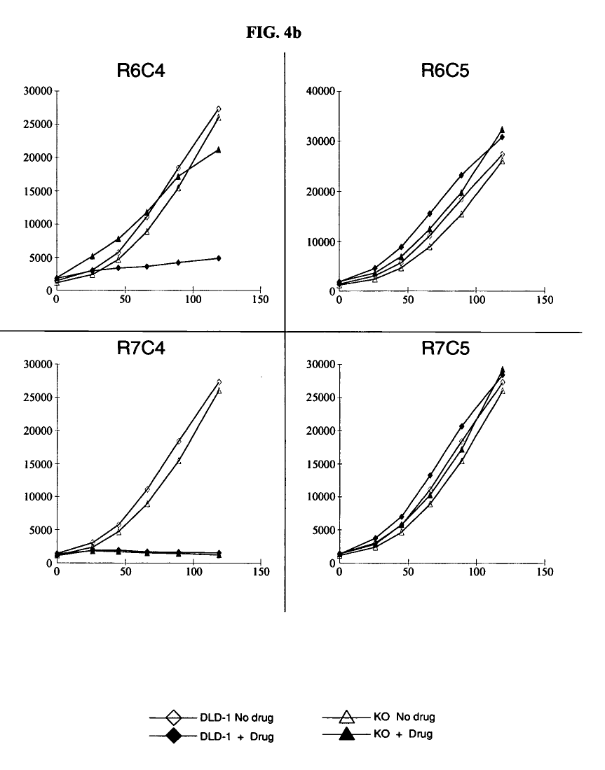

To implement the screen, equal numbers of DLD-1 and KO cells were mixed and distributed in 96 well plates at low dilution. Growth of the two cell types was easily distinguished through fluorescent microscopy (FIG. 3) or high-throughput fluorescence spectroscopy (FIGS. 4a and 4b). In the absence of added drugs, both cell types grew exponentially and equivalently (FIGS. 4a and 4b). Cells could be followed for ˜7 days after plating, at which time the cultures became confluent. Small molecule libraries totaling 29,440 diverse chemical compounds were used in the screen. Each compound was tested in a primary screen at a concentration of 2 μg/ml (Chembridge library) or 2 μM (NCI library). Most compounds at this concentration (27,757) had no effect on the growth of either cell type (for a representative plate see FIG. 4a). 1,683 compounds (5.7%) demonstrated growth inhibitory activity. Several candidates demonstrated selective toxicity for DLD-1 versus KO cells e.g., well # R6C4 (FIG. 4b). However, most compounds were equally toxic both cell lines at this single drug concentration e.g., well # R7C4 (FIG. 4b). In order to identify additional compounds that could be selectively active at lower doses, all 1683 compounds were entered into a secondary screen using a drug concentration range.

183 compounds were shown to possess 2-fold or greater selectivity from the primary and secondary screens. These candidates were subjected to several additional screens. First, each compound was re-tested to confirm the initial results. Second, reproducibly positive compounds were tested at different concentrations to determine the optimum concentration at which differential toxicity to the two lines could be observed. Third, the compounds were tested against other clones of the two cell types, as well as clones which did not express fluorescent proteins, to ensure that the differential toxicity was not the result of clonal variability or confounding effects of the fluorescent proteins or drug resistance genes introduced into the clones tested in the initial screen.

Using this screening procedure, we identified four compounds of interest. Two of these, a wortmannin analog (demethoxyviridin) and mithramycin, have been previously shown to inhibit downstream components of the RAS pathway (Cardenas et al., Trends Biotechnol., 16:427-31, 1998; Campbell et al., Am. J. Med. Sci., 307:167-72, 1994, thereby validating the screening approach. Two additional compounds, TPT and SC-D (see FIG. 2) had novel antiproliferative activity and were evaluated further.

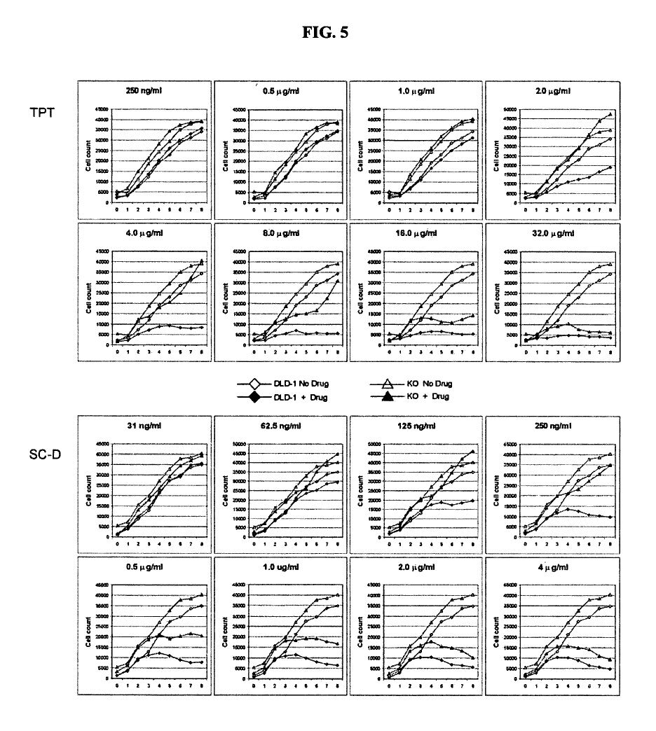

The fluorescence micrographs in FIG. 4 graphically illustrate the inhibition of growth of the mutant c-Ki-Ras containing DLD-1 cells by these two compounds. At the drug concentrations used in this assay, no growth inhibition was observed in the DLD-1 derivatives in which the mutant c-Ki-Ras gene had been disrupted (FIG. 3). Detailed concentration and time course experiments showed that the IC₅₀ for TPT on DLD-1 and KO was ˜2 μg/ml and ˜12 μg/ml, respectively, while the IC₅₀ for SC-D on DLD-1 and KO cells was 125 ng/ml and ˜750 ng/ml, respectively (FIG. 5). Consequently, both compounds demonstrated an approximate 6-fold selectivity for inhibiting the growth of the mutant c-Ki-RAS cell line (DLD-1).

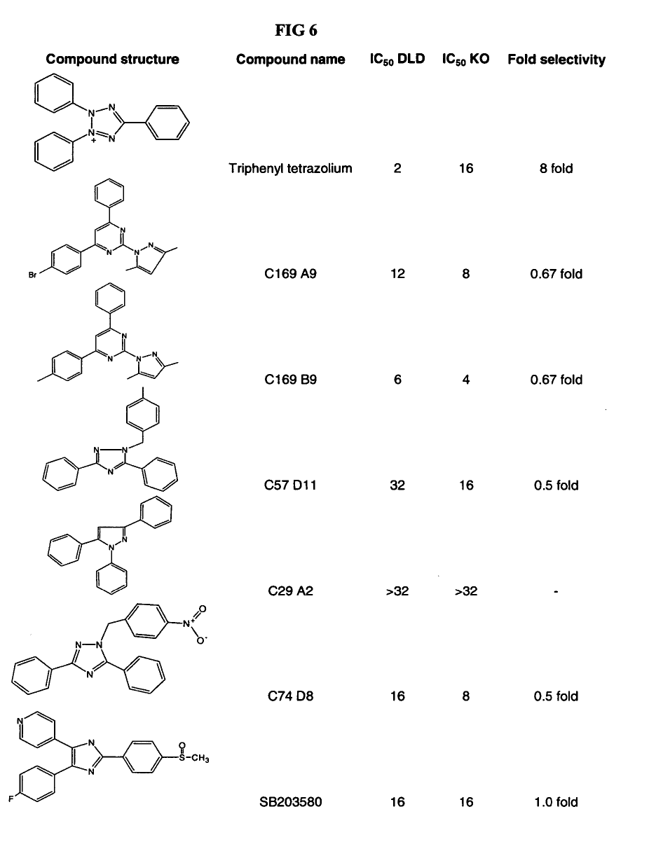

No anti-proliferative activity has previously been described for TPT. Tetrazole compounds such as TPT are commonly used as histological reagents that are reduced by cellular dehydrogenases in viable cells to a coloured formazan dye (Adegboyega et al., Arch. Pathol. Lab. Med., 121:1063-68, 1997, Otero et al., Cytotechnology, 6:137-42, 1991). However, no obvious staining of cells was observed using TPT at the concentrations used in the present assays. Interestingly, several other triphenyl compounds were found in our libraries based on a structural homology search, but none exerted any selectivity towards the mutant RAS-containing line (FIG. 6). A previously identified inhibitor of p38 mitogen activated kinase (SB203580) (Cuenda et al., FEBS Lett., 364:229-33, 1995, Liverton et al., J. Med. Chem., 42:2180-90, 1999) was also noted to be structurally similar to TPT (FIG. 6). However, SB203580 also failed to show selectivity for the mutant-RAS cell line (FIG. 6) and TPT demonstrated little or no reproducible inhibition of p38 or other kinases known to be inhibited by SB203580 (c-Raf-1 and c-Jun kinases) using in vitro kinase assays (data not shown). It is therefore unclear whether kinases represent cellular targets for TPT.

The mechanisms underlying the selective toxicity of SC-D and TPT are unclear. In the past, it has been difficult to determine the actual targets of anti-neoplastic compounds, and the reasons for their selectivity towards cancers are largely obscure (Gibbs, Science, 287:1969-73, 2000). Current models for drug specificity invoke differences in cell cycle parameters, susceptibility to apoptosis, and checkpoint controls. The design of our screen should make future mechanistic studies possible, as we presume that the drugs target some downstream event triggered by the mutant c-Ki-Ras gene.

### EXAMPLE 2

### In Vivo Administration of SC-D in Mouse Model

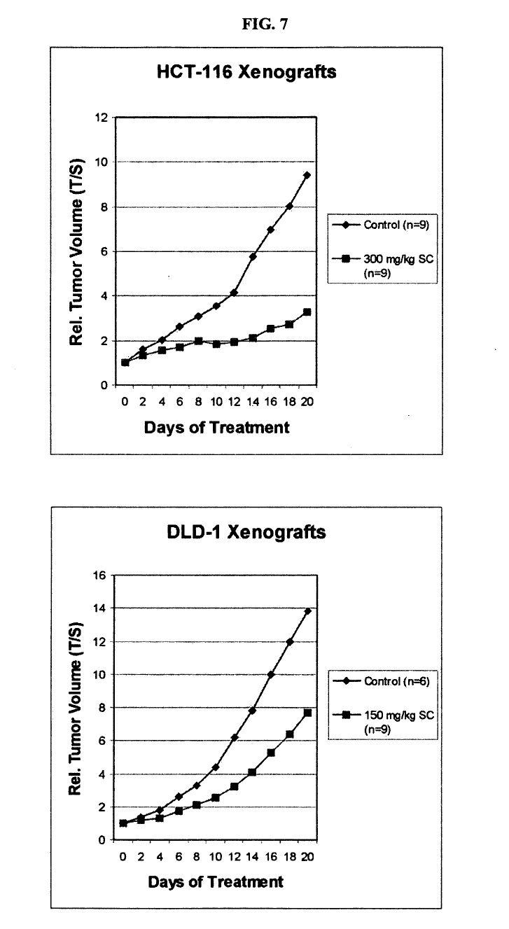

In vivo administration of SC-D was performed to test for specificity for cancer because, in this way, it is possible to compare toxicity to normal cells of diverse types to that of tumors. Two colon cancer cell lines, HCT116 and DLD-1, both of which harbour a single G13D point mutation in the c-Ki-Ras gene, were grown as xenografts in nude mice for testing of SC-D in vivo activity (FIG. 7). Female nude mice, age 4-6 weeks, were implanted with subcutaneous xenografts using DLD-1 or HCT-116 colon cancer cells (5×10⁶ cells). Palpable tumors were established three to six days after cells were injected, at which point drug treatment was initiated. Drugs were administered every day by intraperitoneal injection in a total volume of 400 μl (phosphate buffered saline). Xenografts were measured (major and minor axis) every 2 days using calipers and tumor volume calculated using the equation: length×width²×0.5. Tumor volumes were plotted for control and treated groups by dividing the average tumor volume (T) for each data point by average starting tumor volume (S). DLD-1 tumors were approximately 45% smaller in mice treated with SC-D intraperitoneally for 20 days than in control, untreated mice. HCT-116 tumor growth was inhibited approximately 65% (Students t-test; P<0.05) in animals treated with SC-D for the same time period. These responses exceed the standard criteria for promising lead compounds established by the NCI (Geran et al., Cancer Chemother. Rep., 3:1-103, 1972).

### EXAMPLE 3

### Synthesis of SC-D

SC-D was synthesized as follows: 5′-Dimethoxytritylcytidine (2.72 g, 5 mM) dried in vaccuo for 24 hr, was dissolved in anhydrous tetrahydrofuran (THF) (100 ml) and was cooled to 100° C. Freshly distilled thionyl chloride (365 ml, 5 mM) in anhydrous THF (10 ml) was added dropwise over a period of 30 minutes with stirring. The reaction mixture was warmed to room temperature and stirred at room temperature for an additional 24 hr. The solid which separated was filtered and washed thoroughly with anhydrous THF (4×25 ml). The solid was dried in vaccuo for 24 hr, and the crude product chromatographed on a silica gel column using chloroform and methanol (9:1) as eluant. The parent compound, sulfinyl cytidine was found to be very unstable and hence present only in small quantities. Fractions containing the major degradation product (Rf=0.47 on a silica TLC plate using a chloroform and methanol (75:25) developing system) were combined and concentrated in vaccuo. The product thus obtained was crystallized twice with water and dried in vaccuo for 24 hr to obtain SC-D. Based on mass-spectrometry, SC-D represents a deoxycytidine analogue containing an unsaturated sugar moiety (FIG. 2b).

1. A pair of cells comprising:
a first cell; and
a second cell,
wherein the first cell and the second cell are isogenic but for:
a gene of interest and a gene encoding a fluorescent protein;
wherein the first cell comprises a gene that encodes a first fluorescent protein having a first absorption spectrum and a first emission spectrum;
wherein the second cell comprises a gene that encodes a second fluorescent protein having a second absorption spectrum and a second emission spectrum; and
wherein either:
the first and second absorption spectra are not identical; and/or
the first and second emission spectra are not identical.

2. The pair of cells of claim 1 wherein the first and second absorption spectra are not identical and the first and second emission spectra are not identical.

3. The pair of cells of claim 1 wherein the cells are contained within the same undivided container.

4. The pair of cells of claim 1 wherein the first cell is homozygously wild-type for the gene of interest and wherein the second cell is homozygously mutant for the gene of interest.

5. The pair of cells of claim 1 wherein the gene of interest in the second cell is homozygously deleted.

6. The pair of cells of claim 1 wherein the first cell comprises two wild-type alleles of the gene of interest and wherein the second cell comprises a wild-type allele and a mutant allele of the gene of interest, wherein the mutant allele is dominant.

7. The pair of cells of claim 1 wherein the gene of interest is an oncogene and the first cell is homozygous for a mutant allele of the oncogene and wherein the second cell comprises a homozygous deletion of the mutant oncogene.

8. The pair of cells of claim 1 wherein the first cell expresses the gene of interest and wherein the second cell does not express the gene of interest.

9. The pair of cells of claim 1 wherein the first cell comprises a wild-type allele and a mutant allele of the gene of interest and the second cell is hemizygous for the wild-type allele of the gene of interest.

10. The pair of cells of claim 1 wherein the first cell expresses a protein encoded by the gene of interest and wherein the second cell does not express a protein encoded by the gene of interest.

11. The pair of cells of claim 1 wherein the first and second cells are mammalian cells.

12. The pair of cells of claim 1 wherein the first and second cells are human cells.

13. The pair of cells of claim 1 wherein the cells are cancer cells.

14. The pair of cells of claim 13 wherein the cancer cells are selected from the group consisting of colon tumor cells and breast tumor cells.

15. The pair of cells of claim 1 wherein the cells are HCT116 cells.

16. The pair of cells of claim 1 wherein the cells are DLD-1 cells.

17. The pair of cells of claim 1 wherein the first and second fluorescent proteins are selected from the group consisting of green fluorescent protein, red fluorescent protein, blue fluorescent protein, yellow fluorescent protein, and cyan fluorescent protein.

18. The pair of cells of claim 1 wherein the gene of interest is Ras and wherein the Ras genotype of the first cell is c-Ki-Ras^{WT/mutant} and wherein the Ras genotype of the second cell is c-Ki-Ras^{WT/null}.

19. A pair of cells comprising:
a first cell wherein the Ras genotype of the first cell is c-Ki-Ras^{WT/mutant} and wherein the first cell comprises a first gene that encodes a first fluorescent protein having a first absorption spectrum and a first emisson spectrum; and
a second cell wherein the Ras genotype of the second cell is c-Ki-Ras^{WT/null} and wherein the second cell comprises a second gene that encodes a second fluorescent protein having a second absorption spectrum that is not identical to the first absorption spectrum and a second emission spectrum that is not identical to the first emission spectrum, wherein the first and second cells are isogenic but for the Ras gene and the gene encoding a fluorescent protein.

20. The pair of cells of claim 19 wherein the first fluorescent protein is blue fluorescent protein and the second fluorescent protein is yellow fluorescent protein.

21. A method of making a pair of cells, comprising the steps of:
genetically modifying a first cell to yield a second cell that is isogenic with the first cell but for a single gene of interest;
transfecting the first cell with a first gene that encodes a first fluorescent protein having a first absorption spectrum and a first emission spectrum; and
transfecting the second cell with a second gene that encodes a second fluorescent protein having a second absorption spectrum and a second emission spectrum, wherein either the first and second absorption spectra are not identical and/or the first and second emission spectra are not identical.

22. The method of claim 21 wherein the first and second absorption spectra are not identical and wherein the first and second emission spectra are not identical.

23. The method of claim 21 wherein the first and second cells are mammalian cells.

24. The method of claim 21 wherein the first and second cells are human cells.

25. The method of claim 24 wherein the human cells are human cancer cells.

26. The method of claim 25 wherein the human cancer cells are selected from the group consisting of colon tumor cells and breast tumor cells.

27. The method of claim 21 wherein the first and second cells are HCT116 cells.

28. The method of claim 21 wherein the first and second cells are DLD-1 cells.

29. The method of claim 21 wherein the Ras genotype of the first cell is c-Ki-Ras^{WT/mutant} and wherein the Ras genotype of the second cell is c-Ki-Ras^{WT/null}.

30. A method of identifying a test compound as selectively affecting a gene of interest or its expression products or downstream genes or proteins in its pathway comprising the steps of:
culturing a first and second cell, wherein the first and second cells are isogenic but for a gene of interest and a gene encoding a fluorescent protein, wherein the first cell comprises a first gene that encodes a first fluorescent protein having a first absorption spectrum and a first emission spectrum, and wherein the second cell comprises a second gene that encodes a second fluorescent protein having a second absorption spectrum and a second emission spectrum, wherein either the first and second absorption spectra are not identical and/or the first and second emission spectra are not identical;
contacting the first and second cells with a test compound; and
identifying the test compound as selectively affecting the gene of interest or its expression products or downstream genes or proteins in its pathway if the growth rate of the first cell is altered with respect to the growth rate of the second cell.

31. The method of claim 30 wherein the first and second cells are co-cultured.

32. The method of claim 30 wherein an equal number of the first and second cells are cultured.

33. The method of claim 30, wherein the first and second absorption spectra are not identical and wherein the first and second emission spectra are not identical.

34. The method of claim 30 wherein the fluorescent proteins are detected using fluorescence microscopy to assess growth rate.

35. The method of claim 30 wherein the fluorescent proteins are detected using high-throughput fluorescence spectroscopy to assess growth rate.

36. The method of claim 30 wherein the first cell is homozygously wild-type for the gene of interest and wherein the second cell is homozygously mutant for the gene of interest.

37. The method of claim 30 wherein the gene of interest in the second cell is homozygously deleted.

38. The method of claim 30 wherein the first cell comprises two wild-type alleles of the gene of interest and wherein the gene of interest in the second cell comprises a wild-type allele and a mutant allele of the gene of interest, wherein the mutant allele is dominant.

39. The method of claim 30 wherein the first cell is homozygous for a mutant oncogene and wherein the second cell comprises a homozygous deletion of the mutant oncogene.

40. The method of claim 30 wherein the first cell expresses the gene of interest and wherein the second cell does not express the gene of interest.

41. The method of claim 30 wherein the first cell comprises a wild-type allele and a mutant allele of the gene of interest and wherein the second cell is hemizygous for a wild-type allele of the gene of interest.

42. The method of claim 30 wherein the first cell expresses a mutant protein encoded by the gene of interest and wherein the second cell does not express a protein encoded by the gene of interest.

43. The method of claim 30 wherein the first and second cells are mammalian cells.

44. The method of claim 30 wherein the first and second cells are human cells.

45. The method of claim 30 wherein the cells are cancer cells.

46. The method of claim 45 wherein the cancer cells are selected from the group consisting of colon tumor cells and breast tumor cells.

47. The method of claim 30 wherein the first and second cells are HCT116 cells.

48. The method of claim 30 wherein the first and second cells are DLD-1 cells.

49. The method of claim 30 wherein the first and second fluorescent protein are selected from the group consisting of green fluorescent protein, red fluorescent protein, blue fluorescent protein, yellow fluorescent protein, and cyan fluorescent protein.

50. A method of identifying a test compound as selectively affecting a Ras gene, Ras protein, or downstream gene or protein in its pathway in cells comprising:
contacting a test compound with a co-culture of an essentially equal number of a first and a second cell that are isogenic but for their Ras genes and a gene encoding a fluorescent protein, wherein the Ras genotype of the first cell is c-Ki-Ras^{WT/mutant} and wherein the first cell comprises a first gene encoding a first fluorescent protein having a first absorption spectrum and a first emisson spectrum and wherein the Ras genotype of the second cell is c-Ki-Ras^{WT/null} and wherein the second cell comprises a second gene encoding a second fluorescent protein having a second absorption spectrum that is not identical to the first absorption spectrum and a second emission spectrum that is not identical to the first emission spectrum; and
identifying the test compound as selectively affecting the Ras gene, Ras protein, or downstream gene or protein in the pathway if the growth rate of the first cell is altered with respect to the growth rate of the second cell.

51. The method of claim 50 wherein the first fluorescent protein is blue fluorescent protein and wherein the second fluorescent protein is yellow fluorescent protein.

52. A composition comprising at least 90% of a compound having a formula selected from the group consisting of:

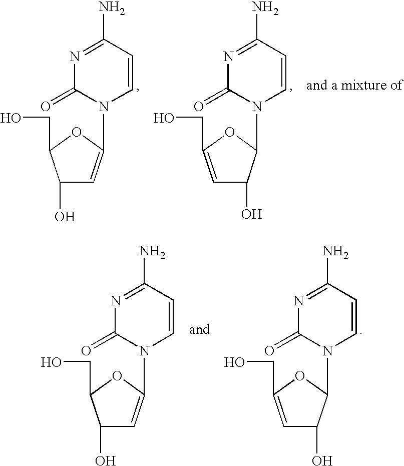

53. A pharmaceutical composition comprising a compound with a formula selected from the group consisting of: 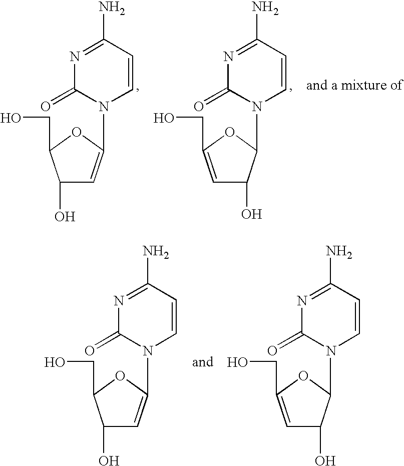
or pharmaceutically acceptable salts, solvates, or prodrugs thereof, and a pharmaceutically appropriate carrier.

54. A cytotoxic composition comprising a compound having a formula: 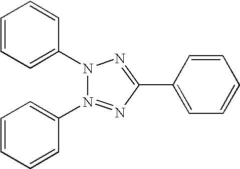
or a pharmaceutically acceptable salt, solvate, or prodrug thereof; and a pharmaceutically appropriate carrier.

52. A method of treating cancer comprising:
administering to a patient in need thereof a therapeutically effective amount of a compound having a formula selected from the group consisting of: 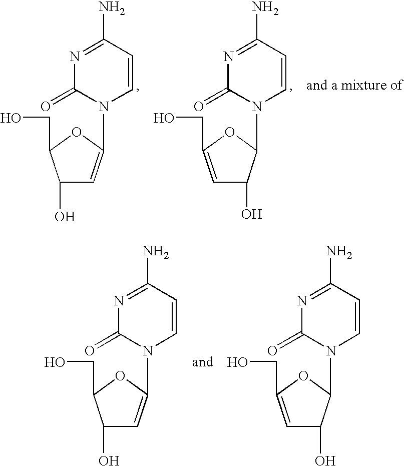
or a pharmaceutically acceptable salt, solvate, or prodrug thereof, and a pharmaceutically appropriate carrier.
